(19) United States
(12) Patent Application Publication
(10) Pub. No.: US 2009/0208922 A1
(43) Pub. Date: Aug. 20, 2009

(54) FET BASED SENSOR FOR DETECTING BIOMOLECULE, METHOD FOR PREPARING THE SAME, AND METHOD FOR DETECTING BIOMOLECULE USING THE FET BASED SENSOR

(76) Inventors: Hee Cheul Choi, Kyung-buk (KR); Hye Ryung Byon, Cambridge, MA (US)

Correspondence Address: HAYNES BEFFEL & WOLFELD LLP, P O BOX 366, HALF MOON BAY, CA, 94019, US

(21) Appl. No.: 12/096,172

(22) PCT Filed: Dec. 5, 2006
(86) PCT No.: PCT/KR2006/005205

§ 371 Date: Mar. 23, 2009

(30) Foreign Application Priority Data
Dec. 5, 2005 (KR) 1020050117687

(51) Int. Cl. G01R 27/02; H01L 21/283; H01L 29/772; C12Q 1/25

(52) U.S. Cl. 435/4; 438/49; 257/253; 324/693; 257/E29.242; 257/E21.159

(57) ABSTRACT

Provided is a SWNT-FET-based sensor for detection of biomolecules including an increased Schottky contact area, a method for preparing the same, and a method for detection of biomolecules using the SWNT-FET-based sensor. According to the method of the present invention, a SWNT-FET-based sensor for detection of biomolecules having a thin and increased Schottky contact area can be obtained. The biomolecule detection sensor exhibits a superior detection sensitivity, and can effectively detect both nonspecific adsorption of biomolecules and specific biomolecule-biomolecule interactions, even at a low concentration of 1 pM, for example.

### BACKGROUND OF THE INVENTION

1. Field of the Invention

The present invention relates to an FET-based sensor for detection of biomolecules, a method for preparing the same, and a method for detection of biomolecules using the FET-based sensor.

2. Description of Related Art

Biochips refer to micro-scale systems for bioanalysis having an immobilized structure of biomolecules such as DNAs, proteins and the like on a small substrate of glass, silicon or nylon. Biochips include various kinds of systems such as DNA chips having immobilized structures of DNAs, protein chips having immobilized structures of proteins, etc. In addition, the biochips may be broadly categorized into microarray chips and microfluidics chips. The microarray chip is a biochip which is capable of analyzing interaction behavior or patterns of biomolecules by arranging and depositing several thousands to several tens of thousands of DNAs or proteins on a surface of the substrate at regular intervals and treating analytes of interest thereon. Typically, mention may be made of DNA chips and protein chips. The microfluidics chip, also called Lab-on-a-chip, is a biochip which is capable of analyzing reaction behavior or patterns between analytes and a variety of chip-immobilized biomolecular probes or sensors by injecting trace amounts of analytes of interest. The DNA chip may be classified into oligonucleotide chips, cDNA chips and PNA chips, depending upon kinds of probes to be immobilized. The oligonucleotide chip technology is a novel approach which is capable of investigating large-scale genetic diversity. This method enables simultaneous detection and identification of numerous genes by attaching large numbers of synthetic oligonucleotides on a precise position in a very tiny space of a supporter and allowing to hybridize the oligonucleotides with very small amounts of target base (nucleotide) sequences. The oligonucleotide chip is expected to make great contributions to drug resistance detection and diagnosis, mutation detection, detection of single nucleotide polymorphism (SNP), disease diagnosis, and genotyping.

Meanwhile, carbon nanotubes (CNTs) are materials in which carbon atoms are positioned in a hexagonal honeycomb-like pattern to create a tube form. CNTs are extremely small materials having a tube diameter in a nanometer size. Depending on the number of tube walls, CNTs can be classified into single-walled carbon nanotubes (SWNTs), multi-walled carbon nanotubes (MWNTs) and rope nanotubes. Since the first discovery of Fullerene (C60), an allotrope of carbon, by Kroto and Smalley in 1985, the carbon nanotubes were discovered in 1991 by Dr. Tijima of NEC Fundamental Research Laboratories. During study of a novel material, Fullerene, Dr. Tijima discovered carbon nanotubes having a thin and long tube-like structure when he conducted studies on carbon deposit formed on a graphite negative electrode via arc discharge by using a transmission electron microscope (TEM). This discovery was published in Nature for the first time. Carbon nanotubes can be produced on an industrial scale by various known methods such as arc-discharge, laser vaporization, Plasma Enhanced Chemical Vapor Deposition, Thermal Chemical Vapor Deposition, vapor phase growth, electrolysis, flame synthesis and the like. Carbon nanotubes have superior mechanical properties, electrical selectivity and excellent field emission properties and are high-efficiency hydrogen storage media.

Since the introduction of an electrical nano-biosensor using a silicon nanowire field-effect transistor (SiNf-FET) device, a great deal of intensive research has been actively undertaken to develop label-free electrical sensing systems using similar types of nano-materials. Particularly, due to high biocompatibilities and well-established device properties, considerable attention has been directed to the single-walled carbon nanotubes (SWNTs) as the feasible and promising candidate of biosensor. A critical issue associated with such a system is sensitivity.

Recently, array-type devices made of n-doped and p-doped nanowires (SiNW) combined with microfluidic channels have been demonstrated as a multiplex biosensor system with a protein detection limit of femtomole concentration level (Zheng, G.; Patolsky, F.; Cui, Y.; Wang, W. U.; Lieber, C. M. Nat. Biotech. 2005, 23, 1294).

However, single-walled carbon nanotube field-effect transistor (SWNT-FET) devices suffer from a low sensitivity, i.e., a reliable detection limit of about 0.1 to 100 nM, for sensing of target proteins or protein-protein interactions ((D Chen, R. J.; Bangsaruntip, S.; Drouvalakis, K. A.; Kam, N. W. S.; Shim, M.; Li, Y.; Kim, W.; Utz, P. J.; Dai, H. Proc. Natl. Acad. Sci. U.S.A. 2003, 100, 4984; and (2) Chen, R. J.; Choi, H. C; Bangsaruntip, S.; Yenilmez, E.; Tang, X.; Wang, Q.; Chang, Y.-L.; Dai, H. J. Am. Chem. Soc. 2004, 126, 1563).

The relatively lower sensitivity of the SWNT devices is intimately correlated to the sensing mechanisms and the corresponding device geometries. Unlike the SiNW devices which perform sensing of protein-protein interactions via chemical gating effects, the SWNT devices are operated by the Schottky barrier (SB) modulation effects as well as by the chemical gating effects. Particularly when an isoelectric point (pi) of the protein becomes close to a pH of reaction media, the Schottky barrier (SB) effects prevalently dominate.

### SUMMARY OF THE INVENTION

Therefore, the present invention has been made in view of the above problems, and it is an object of the present invention to provide a method for preparing an FET-based sensor for detection of biomolecules having a significantly increased sensitivity.

It is another object of the present invention to provide an FET-based sensor for detection of biomolecules, which is prepared by the above-mentioned method.

It is yet another object of the present invention to provide a method for detection of biomolecules using the above-mentioned FET-based sensor for detection of biomolecules.

In accordance with an aspect of the present invention, the above and other objects can be accomplished by the provision of a method for preparing an FET-based sensor for detection of biomolecules, comprising depositing carbon nanotubes on a substrate to form a densely packed network of carbon nanotubes; disposing a shadow mask parallel to and at a distance spaced from the substrate; and irradiating a metal in a tilted angle relative to the vertical plane of the shadow mask, thereby depositing source and drain metal electrodes.

In one embodiment of the present invention, the deposition of carbon nanotubes may be carried out by a method selected from the group consisting of Chemical Vapor Deposition (CVD), laser ablation, arc-discharge, Plasma Enhanced Chemical Vapor Deposition (PECVD), Thermal Chemical Vapor Deposition, vapor phase growth, electrolysis and flame synthesis.

In one embodiment of the present invention, the substrate may be selected from the group consisting of a silicon wafer, a glass, quartz, a metal and a plastic.

In one embodiment of the present invention, the carbon nanotubes may be single-walled carbon nanotubes (SWNTs).

In one embodiment of the present invention, the shadow mask may be a metal or semiconductor thin film.

In one embodiment of the present invention, the shadow mask may have a width of 10 to 2000 μm.

In one embodiment of the present invention, the shadow mask may be disposed at a distance of 30 to 1000 μm spaced from the substrate.

In one embodiment of the present invention, the tilted angle may be in the range of 5 to 35 degrees.

In one embodiment of the present invention, the deposition of metal electrodes may be carried out by Physical Vapor Deposition (PVD), e-beam evaporation or thermal evaporation.

In one embodiment of the present invention, the metal may be at least one selected from the group consisting of platinum (Pt), gold (Au), chromium (Cr), copper (Cu), aluminum (Al), nickel (Ni), palladium (Pd) and titanium (Ti).

In one embodiment of the present invention, the metal may be deposited in a thickness of 15 to 200 run.

In one embodiment of the present invention, the biomolecule may be a nucleic acid or a protein.

In accordance with another aspect of the present invention, there is provided an FET-based sensor for detection of biomolecules having an increased Schottky contact area, which is prepared by the above-mentioned method.

In accordance with yet another aspect of the present invention, there is provided a method for detection of biomolecules, comprising introducing biomolecules into a source electrode surface, a gate surface and a drain electrode surface of the FET-based sensor for detection of biomolecules; and measuring a value of an electric current flowing in a channel region between the source and drain of the sensor.

In one embodiment of the present invention, introduction of the biomolecules may include introducing probe biomolecules into a source electrode surface, a gate surface and a drain electrode surface of the FET-based sensor for detection of biomolecules; and introducing target biomolecules into the source electrode surface, the gate surface and the drain electrode surface of the FET-based sensor.

In one embodiment of the present invention, the biomolecule may be a nucleic acid or a protein.

In one embodiment of the present invention, the nucleic acid may be selected from the group consisting of DNA, RNA, PNA, LNA and hybrids thereof.

In one embodiment of the present invention, the protein may be selected from the group consisting of an enzyme, a substrate, an antigen, an antibody, a ligand, an aptamer and a receptor.

### BRIEF DESCRIPTION OF THE DRAWINGS

The above and other objects, features and other advantages of the present invention will be more clearly understood from the following detailed description taken in conjunction with the accompanying drawings, in which:

FIG. 1A is a schematic view showing a method for preparing an FET-based sensor for detection of biomolecules, according to one embodiment of the present invention, FIG. 1B is a detailed view of the dashed square area in FIG. 1A, and FIG. 1C is an AFM image of a network SWNT prepared in FIG. 1A;

FIGS. 2A and 2B are respectively I-Vg graphs showing pseudo-metallic characteristics of FET fabricated in FIG. 1, and FIGS. 2C and 2D are respectively graphs showing conductance drops upon addition of SpA and SA at various concentrations;

FIG. 3 is a schematic view illustrating the concept of a protein detection method conducted in embodiments of the present invention. 3A: Protein Sensing using a homemade Teflon electrochemical cell; 3B: Nonspecific protein adsorption; and 3C: Specific binding of a target protein to an immobilized probe protein and Tween 20-protected device;

FIG. 4A shows conductance drops of the system upon the specific recognition of SpA by IgG, and FIG. 4B shows conductance drops of the system upon the specific recognition of hCG by anti β-hCG, respectively. Insets: I-Vg curves of the corresponding devices;

FIG. 5A is a schematic view of protein adsorption on a surface of a thick metal electrode using a microsyringe, and FIG. 5B is a graph showing changes in a conductance of a network SWNT-FET device, upon adsorption of PBS and SpA on a thick metal; and

FIG. 6A is an SEM image of a metal electrode fabricated by photolithography, FIG. 6B is an enlarged view of the dashed square area in FIG. 6A, FIG. 6C is an SEM image of a metal electrode fabricated by using a shadow mask, and FIG. 6D is an enlarged view of the dashed square area in FIG. 6C.

### DETAILED DESCRIPTION

Hereinafter, embodiments of the present invention will be described in more detail with reference to the accompanying drawings.

An aspect of the present invention relates to a method for preparing an FET-based sensor for detection of biomolecules, comprising depositing carbon nanotubes on a substrate to form a densely packed network of carbon nanotubes; disposing a shadow mask parallel to and at a distance spaced from the substrate; and irradiating a metal in a tilted angle relative to the vertical plane of the shadow mask, thereby depositing source and drain metal electrodes.

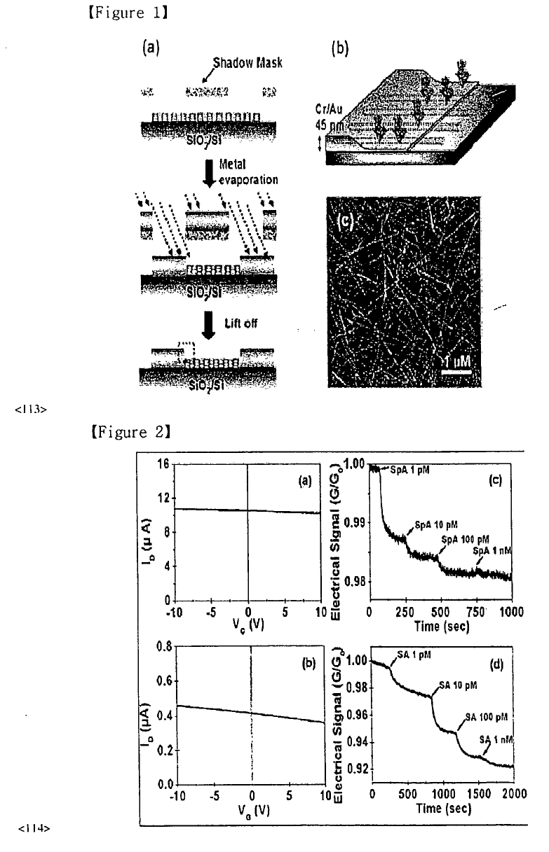

FIG. 1A schematically shows a method for preparing an FET-based sensor for detection of biomolecules, according to one embodiment of the present invention.

Referring to FIG. 1, in order to prepare a biomolecule detection sensor, carbon nanotubes are first deposited on a substrate to form a densely packed network of carbon nanotubes. Although the carbon nanotubes may be single-walled carbon nanotubes (SWNTs), multi-walled carbon nanotubes (MWNTs) or rope nanotubes, preferred are SWNTs. In addition, the substrate may be selected from the group consisting of a silicon wafer, a glass, quartz, a metal and a plastic. Preferred is the silicon wafer.

Deposition of the carbon nanotubes may be carried out by a conventional method known in the art. For example, deposition of the carbon nanotubes may be performed by a method selected from the group consisting of Chemical Vapor Deposition (CVD), laser ablation, arc-discharge, Plasma Enhanced Chemical Vapor Deposition (PECVD), Thermal Chemical Vapor Deposition, vapor phase growth, electrolysis and flame synthesis.

In FIG. 1, single-walled carbon nanotubes were deposited on a SiO2/Si substrate by CVD. FIG. 1C shows an AFM image of a network SWNT prepared in FIG. 1A.

As a next step to prepare the biomolecule detection sensor, a shadow mask is disposed parallel to and at a distance spaced from the substrate.

There is no particular limit to a material for the shadow mask, and therefore the shadow mask may be fabricated using any material that is conventionally used in the art. For example, the shadow mask may be a metal or semiconductor thin film.

The shadow mask may have a certain width. A length of the SWNT channel of the fabricated device is determined by the width of the shadow mask and may be a proper size which can be sufficiently included in the fabricated electrochemical cell while not causing short-circuiting of both electrodes by the irradiated metal. For example, the shadow mask may have a width of 10 to 2000 μm.

The shadow mask may be disposed at a given distance spaced from the substrate, for example a distance of 30 to 1000 μm. However, there is no particular limit to the spaced distance.

The spaced distance corresponds to a thickness of carbon tapes attached to both upper ends of the SiO2/Si wafer, respectively, during the FET fabrication process, and the spacing between the wafer and shadow mask is maintained at a predetermined distance, based on the carbon tapes. Therefore, the spaced distance requires a length to ensure that both electrodes of the source and drain are not completely short-circuited, taking into consideration the deposition angle of the irradiated metal and the width of the shadow mask together with the spaced distance.

Next, a metal is irradiated in a tilted angle relative to the vertical plane of the shadow mask, thereby depositing source and drain metal electrodes.

There is no particular limit to a metal deposition angle. For example, the deposition angle may be in the range of 5 to 35 degrees.

It is enough that if the metal deposition angle does not result in complete short-circuiting of both electrodes of the source and drain, taking into consideration the spaced distance and width of the shadow mask together with the deposition angle. In other words, there is a close relationship between the above-mentioned three factors, i.e. the metal deposition angle, the spaced distance of the shadow mask and the width of the shadow mask, and the value of each factor may be appropriately controlled depending upon the values of the remaining factors. For example, where the width of the shadow mask becomes broader, the spaced distance of the shadow mask may be slightly increased or the deposition angle may be slightly further tilted, so long as both electrodes do not undergo short-circuiting.

Deposition of the metal electrodes may be carried out by a conventional method known in the art, for example, Physical Vapor Deposition (PVD), e-beam evaporation or thermal evaporation. In addition, the metal may be at least one selected from the group consisting of platinum (Pt), gold (Au), chromium (Cr), copper (Cu), aluminum (Al), nickel (Ni), palladium (Pd) and titanium (Ti).

Referring back to FIG. 1A, for irradiation of the metal in an angle of range of 5 to 35 degrees relative to the vertical plane of the shadow mask, the metal irradiation was carried out using a thermal evaporator equipped with a 23 degree-tilted sample stage. During deposition of Cr (15 nm) followed by Au (30 nm), metals were guided to penetrate underneath the shadow mask.

FIG. 1B is a detailed view of the dashed square area in FIG. 1A. Referring to FIG. 1B, it can be seen that a thin and wide Schottky contact area was formed. There is no particular limit to a thickness of the deposited metal. The metal may be deposited in a thickness of 15 to 200 nm.

FIGS. 2A and 2B are respectively I-Vg graphs showing pseudo-metallic characteristics of FET fabricated in FIG. 1. Referring to FIGS. 2A and 2B, the fabricated FET exhibited very little gate field dependence, i.e. pseudo-metallic transport characteristics. Such abnormal transport characteristics represent that a thin and wide metal coating, i.e. an increased Schottky contact area was obtained.

Apparently from FIGS. 2A and 2B, it may be thought that all of the SWNTs are metals. However, network SWNTs, grown by the CVD method but fabricated into the FET device by photolithography and e-beam lithography, are composed of semiconducting and metallic nanotubes in a suitable ratio, and this leads to a conductance drop of more than 50% upon changes of an electrostatic gate field in a voltage range of −10 to 10 V (Choi, H. C; Kundaria, S.; Wang, D.; Ajavey, A.; Wang, Q.; Rolandi, M.; Dai, H. Nano Lett. 2003, 3, 157).

Further, a similar metallic transport phenomenon may also be observed even when complete short-circuiting of the source and drain electrodes occurs due to severe metal penetration. However, when FET having no SWNTs is fabricated using a similar method, the probability of electrode short-circuiting can be easily eliminated because a negligible quantity of current flow, i.e. 20 pA, is observed. Therefore, it is preferably understood that the pseudo-metallic characteristics of the FET device fabricated using the above-mentioned shadow mask are due to penetrated metals which form a thin and wide metal coating on the SWNT channels.

Another aspect of the present invention relates to an FET-based sensor for detection of biomolecules having an increased Schottky contact area, which is prepared by the above-mentioned method.

As discussed hereinbefore, the thin and wide Schottky contact area can be formed via deposition of source and drain metal electrodes by irradiating a metal in a tilted angle of 5 to 35 degrees relative to the vertical plane of the shadow mask. The detection limit concentration of biomolecules can be significantly lowered by the thin and wide Schottky contact area.

Yet another aspect of the present invention relates to a method for detection of biomolecules, comprising introducing biomolecules into a source electrode surface, a gate surface and a drain electrode surface of the FET-based sensor for detection of biomolecules; and measuring a value of an electric current flowing in a channel region between the source and drain of the FET-based sensor. This method is directed to detection of nonspecific binding of biomolecules.

There is no particular limit to kinds of the biomolecules. For example, the biomolecule may be a nucleic acid or a protein. The nucleic acid may be selected from the group consisting of DNA, RNA, PNA, LNA and hybrids thereof, and the protein may be selected from the group consisting of an enzyme, a substrate, an antigen, an antibody, a ligand, an aptamer and a receptor.

Introduction of the biomolecules may include introducing probe biomolecules into a source electrode surface, a gate surface and a drain electrode surface of the FET-based sensor for detection of biomolecules; and introducing target biomolecules into the source electrode surface, the gate surface and the drain electrode surface of the FET-based sensor. This is intended for detection of specific binding between the biomolecules.

The biomolecule detection method according to the present invention is characterized in that the biomolecules are introduced into the source and drain electrode surfaces as well as the gate surface.

In one embodiment of the present invention, nonspecific protein adsorption and specific protein-protein bindings were detected using the detection sensor prepared as above.

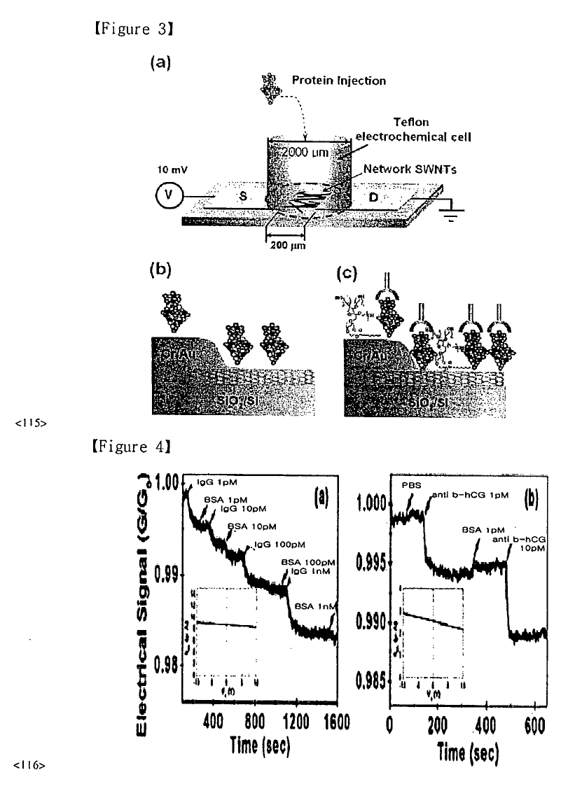

FIG. 3 is a schematic view illustrating the concept of a protein detection method conducted in embodiments of the present invention. 3A: Protein Sensing using a homemade Teflon electrochemical cell; 3B: Nonspecific protein adsorption; and 3C: Specific binding of a target protein to an immobilized probe protein and Tween 20-protected device.

Referring to FIG. 3, it can be seen that the Teflon electrochemical cell has a reaction area having a diameter of about 2 to 3 mm, which is designed to expose both channels and electrodes of the transistor to a buffer solution and proteins.

Returning to FIG. 3, the biomolecule detection sensor is installed into the electrochemical cell which was then filled with a phosphate-buffered solution (PBS, 10 mM, pH=7.4) while continuously applying a bias voltage (Vds) of 10 mV between the source and drain electrodes. When the device started to exhibit a steady current, proteins at specific concentrations were injected into the cell using a micropipet.

In order to compare the detection sensitivity of the devices according to the present invention with that of the ones fabricated by photolithography, nonspecific adsorption was examined for the same proteins used in previous reports, such as Protein A (SpA, derived from Staphylococcus aureus, Zymed®), Streptavidin (SA, Sigma), mouse antibody β-hCG (anti β-hCG, Lab Vision), human chorionic gonadotropin (hCG, Sigma), and rabbit immunoglobulin G (IgG, Sigma).

FIGS. 2C and 2D are graphs showing conductance drops upon addition of SpA and SA, respectively, at various concentrations.

Referring to FIGS. 2C and 2D, all of the examined devices have shown significant conductance drops upon nonspecific adsorption of proteins at as low as 1 pM concentration. Although slightly different extents of conductance drop have been observed from devices to devices, all of the devices have produced reliable and reproducible results.

Further, the increased sensitivity was also confirmed from the specific bindings of protein pairs. Probe proteins were immobilized on the devices by immersing the fabricated devices into the concentrated probe protein solutions for 3 hours, followed by treatment with Tween 20 (0.05 wt % in a PBS solution) for 2 hours. The Tween 20 treatment protects probe protein-unoccupied sites of the device from nonspecific bindings (see FIG. 3C). Once the devices were stabilized, various concentrations of target proteins were injected stepwise.

FIG. 4A shows conductance drops of the system upon the specific recognition of SpA by IgG, and FIG. 4B shows conductance drops of the system upon the specific recognition of hCG by anti β-hCG, respectively. Inset graphs are I-Vg curves of the corresponding devices.

Referring to FIG. 4, the devices show apparent conductance drops at a 1 pM concentration of target proteins. From the fact that injections of PBS and bovine serum albumin (BSA) as control groups did not lead to changes of the conductance (see FIG. 4), it can be seen that the conductance drops are solely attributed to the specific bindings between the probe and the target proteins. It should be noted that the similar types of FET devices fabricated by photolithography or devices fabricated by using the shadow mask without a sample gradient generally detect the specific protein bindings at a concentration of >10 nM ((D Chen, R. J.; Bangsaruntip, S.; Drouvalakis, K. A.; Kam, N. W. S.; Shim, M.; Li, Y.; Kim, W.; Utz, P. J.; Dai, H. Proc. Natl. Acad. Sci. U.S.A. 2003, 100, 4984 (2) Chen, R. J.; Choi, H. C; Bangsaruntip, S.; Yenilmez, E.; Tang, X.; Wang, Q.; Chang, Y.-L.; Dai, H. J. Am. Chem. Soc. 2004, 126, 1563).

In other words, the devices with an increased Schottky contact area have shown a high sensitivity with a 1 pM detection limit for nonspecific bindings of proteins as well as specific bindings of protein pairs. This is a 104-fold increased detection limit as compared to that of the reported similar devices.

The substantially increased sensitivity is primarily due to the increased thin and wide Schottky contact area which is capable of accommodating relatively larger numbers of proteins at a low concentration, thus resulting in prompt modulation of the metal work function of the devices.

In addition, a thickness of the metal covering SWNTs within the Schottky contact area is also an important factor which may affect the sensitivity. The thickness of the metal should be sufficiently thin to an extent that changes of the work function by the protein adsorption can be immediately transmitted to the interface where the Schottky contact is formed.

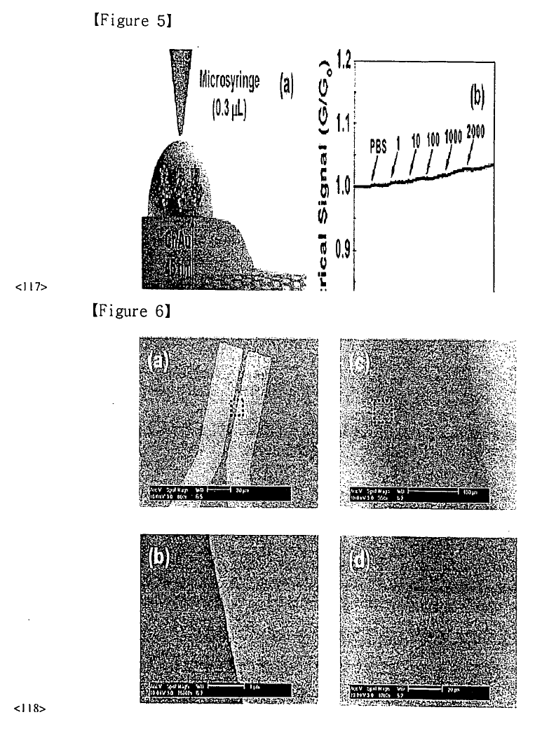

FIG. 5A is a schematic view of protein adsorption on a surface of a thick metal electrode using a microsyringe, and FIG. 5B is a graph showing changes in a conductance of a network SWNT-FET device, upon adsorption of PBS and SpA on a thick metal.

Referring to FIG. 5, there were substantially no changes of the conductance in a control group study conducted by supplying micro-droplets of PBS and proteins to thick metal surfaces which are 45 nm vertically away from the Schottky contact interface. Similar results were also obtained even at very high protein concentrations (>mM).

FIG. 6A is an SEM image of a metal electrode fabricated by photolithography, FIG. 6B is an enlarged view of the dashed square area in FIG. 6A, FIG. 6C is an SEM image of a metal electrode fabricated by using a shadow mask, and FIG. 6D is an enlarged view of the dashed square area in FIG. 6C.

Even though it may be technically difficult to measure the thickness of the penetrated metal film, it can be seen that upon referring to FIG. 6, edges of the metal electrode fabricated by photolithography are sharp and definite, whereas edges of the metal electrode deposited using a shadow mask at a tilted angle is unclearly defined, with a gradient thickness decreasing toward the center of the source and drain electrodes.

In conjunction with the increased Schottky contact area, internanotube Schottky contacts further increases the sensitivity. Since the network SWNTs, grown at a high efficiency by the CVD method, are composed of both semiconducting and metallic nanotubes, the Schottky point contacts are formed in a high density at points where semiconducting and metallic SWNTs are crossed.

As apparent from the above description, a preparation method of the present invention can provide a SWNT-FET-based sensor for detection of biomolecules having a thin and increased Schottky contact area. The biomolecule detection sensor of the present invention exhibits a superior detection sensitivity, and for example, can effectively detect both nonspecific adsorption of biomolecules and specific biomolecule-biomolecule interactions, even at a low concentration of 1 pM, for example.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

We claim:

1. A method for preparing an FET-based sensor for detection of biomolecules, comprising:
depositing carbon nanotubes on a substrate to form a densely packed network of carbon nanotubes;
disposing a shadow mask parallel to and at a distance spaced from the substrate; and
irradiating a metal in a tilted angle relative to the vertical plane of the shadow mask, thereby depositing source and drain metal electrodes.

2. The method according to claim 1, wherein the deposition of the carbon nanotubes is carried out by a method selected from the group consisting of Chemical Vapor Deposition (CVD), laser ablation, arc-discharge, Plasma Enhanced Chemical Vapor Deposition (PECVD), Thermal Chemical Vapor Deposition, vapor phase growth, electrolysis and flame synthesis.

3. The method according to claim 1, wherein the substrate is selected from the group consisting of a silicon wafer, a glass, quartz, a metal and a plastic.

4. The method according to claim 1, wherein the carbon nanotubes are single-walled carbon nanotubes (SWNTs).

5. The method according to claim 1, wherein the shadow mask is a metal or semiconductor thin film.

6. The method according to claim 1, wherein the shadow mask has a width of 10 to 2000 μm.

7. The method according to claim 1, wherein the shadow mask is disposed at a distance of 30 to 1000 μm spaced from the substrate.

8. The method according to claim 1, wherein the tilted angle is in the range of 5 to 35 degrees.

9. The method according to claim 1, wherein the deposition of the metal electrodes is carried out by Physical Vapor Deposition (PVD), e-beam evaporation or thermal evaporation.

10. The method according to claim 1, wherein the metal is at least one selected from the group consisting of platinum (Pt), gold (Au), chromium (Cr), copper (Cu), aluminum (Al), nickel (Ni), palladium (Pd) and titanium (Ti).

11. The method according to claim 1, wherein the metal is deposited in a thickness of 15 to 200 nm.

12. The method according to claim 1, wherein the biomolecule is a nucleic acid or a protein.

13. An FET-based sensor for detection of biomolecules having an increased Schottky contact area, which is prepared by the method of claim 1.

14. A method for detection of biomolecules, comprising:
introducing biomolecules into a source electrode surface, a gate surface and a drain electrode surface of the FET-based sensor for detection of biomolecules according to claim 13; and
measuring a value of an electric current flowing in a channel region between the source and drain of the FET-based sensor.

15. The method according to claim 14, wherein introduction of the biomolecules includes:
introducing probe biomolecules into a source electrode surface, a gate surface and a drain electrode surface of the FET-based sensor for detection of biomolecules; and
introducing target biomolecules into the source electrode surface, the gate surface and the drain electrode surface of the FET-based sensor.

16. The method according to claim 14, wherein the biomolecule is a nucleic acid or a protein.

17. The method according to claim 16, wherein the nucleic acid is selected from the group consisting of DNA, RNA, PNA, LNA and hybrids thereof.

18. The method according to claim 16, wherein the protein is selected from the group consisting of an enzyme, a substrate, an antigen, an antibody, a ligand, an aptamer and a receptor.
